(19) United States
(12) Patent Application Publication
(10) Pub. No.: US 2011/0189201 A1
(43) Pub. Date: Aug. 4, 2011

(54) MODULATION OF BONE DEVELOPMENT

(76) Inventor: REGINALD GORCZYNSKI, Willowdale (CA)

(21) Appl. No.: 13/084,009

(22) Filed: Apr. 11, 2011

Related U.S. Application Data
(63) Continuation of application No. 12/394,621, filed on Feb. 27, 2009.
Application No. 12/394,621 is a continuation of application No. 10/515,332, filed on Jul. 8, 2005, now abandoned.
(60) Provisional application No. 60/386,564, filed on Jun. 7, 2002.

(51) Int. Cl. A61K 38/02; A61K 39/395; A61K 31/713; C12Q 1/02; C12N 5/00; A61P 19/10; A61P 19/02

(52) U.S. Cl. 424/172.1; 514/16.7; 514/44 A; 514/16.9; 435/29; 435/375

(57) ABSTRACT

Methods and compositions for modulating bone development are described.

### FIELD OF THE INVENTION

The present invention relates to methods and compositions for modulating bone development.

### BACKGROUND OF THE INVENTION

The receptor activator of nuclear factor-kappaB ligand (RANKL) also referred to as tumor necrosis factor-related activation-induced cytokine (TRANCE), osteoprotegerin ligand (OPGL—the nomenclature used herein), and osteoclast differentiation factor (ODF), is a transmembrane ligand expressed in osteoblasts and bone marrow stromal cells and produced by T cells (1-4). Following binding to RANK, the osteoclast receptor vital for osteoclast differentiation, activation and survival, OPGL induces osteoclastogenesis in a pathway which synergizes with signals derived from M-CSF (CD98) (5, 6). OPG (or osteoclast inhibitor factor, OCIF), also produced by osteoblasts and marrow stromal cells, lacks a transmembrane domain and acts as a decoy receptor for OPGL, competitively inhibiting binding of OPGL with RANK and thus regulating bone metabolism (7). The crucial role played by OPGL/OPG in regulating bone metabolism is supported by the findings of extremes of skeletal phenotypes (osteoporosis versus osteopetrosis) in mice with altered expression of these molecules, and recent reports that polymorphisms in the osteoprotegerin gene in human are associated with osteoporotic fractures (43). Secretion of OPG and OPGL from osteoblasts and stromal cells is regulated by numerous hormones and cytokines, often in a reciprocal manner (8).

As an example of the complex role of cytokine mediated regulation, TNFα has been documented both to cooperate with OPGL in the generation of osteoclasts in stromal cell-depleted rat bone marrow cell cultures (9), and to inhibit osteoblast differentiation and osteocalcin/bone nodule formation (10) at a stage downstream of signals provided by insulin-like growth factor I (IGF-I) or the osteogenic bone morphogenic proteins (BMPs-2, -4, and -6). The cytokine TGFβ1 is also reported to inhibit BMP-2 induced osteoblast development (11) while, in contrast, TGFβ2 and TGFβ3 both inhibit osteoclastogenesis by increasing OPG (and decreasing OPGL) levels, in association with altered expression of transcription factors Smad and Cbfa1 (12). Enhanced IL-6 production (and enhanced OPGL) is thought to explain the increased osteoclastogenesis and bone loss following chronic alcohol ingestion in mice (13), and similarly IL-4 and IL-13 inhibit proliferation and stimulate IL-6 formation in human osteoblasts(14). An important role for other proinflammatory cytokines (including IL-1) in bone turnover is also well documented (15). Somewhat surprisingly, perhaps, IFNγ, an inflammatory-type cytokine, has been reported to enhance osteoblast activity by countering RANKL-induced osteoclast activation (16).

In terms of hormonal regulation of bone turnover, estrogen (E2) has been shown to modify osteoclast differentiation/activation induced by OPGL by downregulating the activation of Jun N-terminal kinase 1 (JNK1), which in turn decreases nuclear levels of the key osteoclastogenic transcription factors, c-Fos and c-Jun (17). An additional mechanism by which estrogen deficiency enhances osteoclastogenesis may reflect the loss of E2 suppression of T cell production of TNFα (18). Parathyroid hormone (PTH) stimulates osteoclastogenesis, at least in part by inhibiting induction of OPG (19).

In previous studies, the inventor has reported on the functional effect of perturbation of the interactions of the receptor:ligand pair, CD200:CD200 receptor, expressed by cells of the monocyte/myeloid lineage, on cytokine production, particularly the cytokines, IL-6, TNFα and TGFβ (Gorczynski, Europ. J. Immunol. 2001, 31: 2331-2335).

### SUMMARY OF THE INVENTION

The present inventor has shown that incubating bone marrow stromal cells in the presence of either antibodies to a CD200 receptor (CD200R) or a soluble form of CD200 increased the expression of mRNA for various markers of increased bone metabolism. The results indicate, in one respect, that CD200R agonists inhibit the growth and proliferation of osteoclasts which mediate bone resorption. The CD200:CD200R signaling cascade thus is influential in the development of bone cells and tissue, and can be manipulated for therapeutic and other purposes using modulators of the CD200:CD200R interaction.

Accordingly, the present invention provides a method of modulating bone development comprising administering an effective amount of an agent that modulates a CD200 receptor to a cell or animal in need thereof.

In one aspect, the present invention provides a method of stimulating bone development comprising administering an effective amount of a CD200 receptor agonist to a cell or animal in need thereof. Preferably, the CD200 receptor agonist is a CD200 protein or an antibody to the CD200 receptor.

In another aspect, the present invention provides a method of inhibiting bone development comprising administering an effective amount of CD200 receptor antagonist.

In yet another aspect, the present invention includes screening methods for identifying substances which are capable of modulating bone development by modulating CD200 receptors. In particular, the methods may be used to identify substances which are capable of binding to and augmenting or attenuating the effects of CD200 or the CD200 receptors (i.e. agonists). Alternatively, the methods may be used to identify substances which are capable of binding to CD200 receptor and which inhibit the effects of CD200 or a CD200 receptor (i.e. antagonists).

Accordingly, the invention provides a method of identifying substances which bind with a CD200 receptor, comprising the steps of:

(a) incubating a bone cell expressing a CD200 receptor and a test substance, under conditions which allow for formation of a complex, and

(b) assaying for complexes of the CD200 receptor and the test substance, for free substance, and for non-complexed CD200 receptor, wherein the presence of complexes indicates that the test substance is capable of binding the CD200 receptor.

The invention also includes screening assays for identifying agonists or antagonists of a CD200R comprising the steps of:

(a) incubating a test compound with a bone cell expressing a CD200R; and

(b) determining the effect of the compound on the CD200 receptor activity or expression and comparing with a control (i.e. in the absence of the test compound), wherein a change in the CD200 receptor activity or expression as compared to the control indicates that the test compound may modulate bone development.

In a preferred embodiment, the CD200R used in the screening method is on a bone cell that produces a CD200 receptor that yields a measurable marker upon stimulation with CD200.

The present invention also includes the pharmaceutical compositions comprising any of the above molecules that modulate CD200 receptors for use in modulating bone development. The pharmaceutical compositions preferably comprise a CD200 peptide, preferably CD200:Fc, or nucleic acid encoding a CD200 peptide. The pharmaceutical compositions can further comprise a suitable diluent or carrier.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 is a bar graph showing quantitative expression of bone-associated mRNAs in cultured stromal cells with and without LPS.

FIG. 2 is a bar graph showing the production of different cytokines in cultured stromal cells with and without LPS.

FIG. 3 is a bar graph showing absolute concentrations of mRNAs relative to control (1.0 for GAPDH) in mixed cultures of stromal cells and MC3T3, incubated in the presence/absence of anti-CD200R mAb (10 μg/ml).

FIG. 4 is a bar graph showing absolute concentrations of mRNAs relative to control (1.0 for GAPDH) in mixed cultures of stromal cells and MC3T3, incubated with/without murine CD200Fc (1 mg/ml).

FIG. 5 is a bar graph showing expression of different cytokines/chemokines in supernatants of cultures shown in FIG. 3. Values shown represent arithmetic means±SD for 3 individual samples for each time point. ELISA assays were quantitated using recombinant cytokine/chemokine.

FIG. 6 is a bar graph showing expression of different cytokines/chemokines in supernatants of cultures shown in FIG. 4. Again all values shown represent arithmetic means±SD for 3 individual samples for each time point.

### DETAILED DESCRIPTION OF THE INVENTION

### I. Modulation of Bone Development

As previously stated, the present inventor has demonstrated that both a soluble form of CD200 and antibodies to a CD200 receptor (CD200R) have the effect of altering the expression of molecules involved in bone metabolism. In one respect, the CD200R agonists noted above have the effect of inhibiting the expression of genes associated with osteoclastogenesis, and thus are useful to inhibit the growth and proliferation of osteoclasts that are responsible for the resorption of bone. In another respect, these CD200R agonists also result in elevation of markers associated with the growth and proliferation of osteoblasts, which are responsible for the formation of new bone.

Accordingly, the present invention provides a method of modulating bone development by administering an effective amount of an agent that modulates a CD200 receptor to a cell or animal in need thereof. The present invention also provides a use of an effective amount of an agent that modulates a CD200 receptor to modulate bone development. The present invention further provides a use of an effective amount of an agent that modulates a CD200 receptor for the manufacture of a medicament to modulate bone development.

The term “CD200 receptor” as used herein includes any member of the CD200 receptor family from any species including the receptors disclosed in WO 00/70045, WO 02/095030 or GenBank Accession numbers NM170780, NM138940, NM138939, NM138806, NT005612, XM293600, NM010818, AF497550, AF497549, AF497548, AF495380, as well as analogs and homologs of any CD200 receptor.

The term “agent that modulates a CD200 receptor” includes any agent that can stimulate or activate the receptor (e.g. CD200 receptor agonists) as well as any agent that can inhibit or suppress the receptor (e.g. CD200 receptor antagonists). Agents that modulate a CD200 receptor include agents that modulate the interaction of CD200 with a CD200 receptor. Specific examples of CD200 receptor modulators are given in Section

The term “modulate bone development” as used herein refers to the inhibition or suppression as well as the activation or stimulation of the formation, differentiation or development of bone tissue or bone cells such as osteoblasts, osteoclasts, osteocytes, chondroclasts, chondrocytes, chondroclasts or bone marrow cells, including mesenchymal stem and progenitor cells.

The term “effective amount” as used herein means an amount effective, at dosages and for periods of time necessary to achieve the desired results (e.g. the modulation of bone development). Effective amounts of a molecule may vary according to factors such as the disease state, age, sex, weight of the animal. Dosage regime may be adjusted to provide the optimum therapeutic response. For example, several divided doses may be administered daily or the dose may be proportionally reduced as indicated by the exigencies of the therapeutic situation.

The term “animal” as used herein includes all members of the animal kingdom which express CD200, preferably humans.

The term “a cell” includes a single cell as well as a plurality or population of cells. Administering an agent to a cell includes both in vitro and in vivo administrations.

Other immunomodulatory molecules may be used with a CD200R modulator in order to modulate bone development including, but not limited to, cytokines, MD-1 and fg12. A preferred cytokine is IL-11 which is also useful in modulating bone development. Similarly, the CD200R modulator can be used in combination with any other agent useful to modulate bone cell or tissue metabolism, including anabolic agents such as parathyroid hormone such as PTH(1-84), and analogs and active fragments including PTH(1-34), and the bone morphogenetic proteins (BMPs) as well as bone resorption inhibitors including calcitonin, the bisphosphonates, estrogen analogs and receptor modulators, and the like. In addition, vitamin D which acts on osteoclasts, or M-CSF/CSF may be co-administered.

In one aspect, the present invention provides a method of stimulating bone development comprising administering an effective amount of a CD200 receptor agonist to a cell or animal in need thereof. The present invention also includes a use of a CD200 receptor agonist to stimulate bone development. The present invention further includes a use of a CD200 receptor agonist for the manufacture of a medicament to stimulate bone development.

Any CD200R agonist that is useful in stimulating bone development can be used including, but not limited to, antibodies, peptide mimetics, small molecules, CD200 proteins and fragments thereof, soluble CD200, CD200R proteins and fragments thereof, soluble CD200Rs and modulators identified according to the screening assays described herein. In one embodiment, the CD200R agonist is a CD200 protein such as a soluble CD200 protein. In another embodiment, the CD200R agonist is an antibody that crosslinks a CD200 receptor such as a whole anti-CD200 receptor immunoglobulin.

Stimulation of bone development with a CD200R agonist has utility in a wide range of therapeutic applications including any condition wherein one would want to increase the production of bone. Such disorders include, but are not limited to, disorders caused by increased osteoclastogenesis or bone loss associated with inflammatory conditions, infection, injury, genetic disorders and aging such as osteoporosis, osteogenesis imperfecta, osteopenia, Paget's disease, metastatic bone cancer, myeloma bone disease, and bone fracture healing, bone graft repair, and including disorders associated not only with skeletal conditions but also those associated with dental conditions.

In another aspect, the present invention provides a method of inhibiting bone development comprising administering an effective amount of CD200 receptor antagonist. The present invention also includes a use of a CD200R antagonist to inhibit bone development or for the manufacture of a medicament to inhibit bone development.

The CD200R antagonist can be any agent that can block the activation or stimulation of a CD200R such as an antibody fragment, small molecule, peptide mimetic, peptide or an antisense oligonucleotide to a CD200 receptor. In one embodiment, the antagonist is an antibody fragment that binds to the CD200 receptor but does not activate or crosslink the receptor. In a specific embodiment, the antibody fragment is an F(ab′)₂ or Fab fragment

There are many conditions wherein one might want to prevent bone development. Preferably in disorders caused by decreased osteoclastogenesis or increased bone mass associated with inflammatory conditions, infection, malignancies, injury, genetic disorders and aging, including osteopetrosis and fibrous dysplasia as well as genetic and connective tissue hyperostosis conditions.

### II. CD200R Modulators

Any agent that can modulate a CD200 receptor and modulate bone development can be used in the methods and compositions of the invention. Some CD200R modulators are further described below.

### (a) CD200 or CD200 Receptors

A CD200 protein or CD200 receptor molecule may be used as a CD200 receptor modulator. A CD200 protein would act as a CD200 receptor agonist while a CD200R protein may act as a CD200 receptor antagonist. CD200 or CD200R proteins may be obtained from known sources or prepared using recombinant DNA techniques. The sequence of CD200 (previously known as OX-2) may be obtained from GenBank (human, M17226, X0523; rat, X01785; mouse, AF029214). The sequence of several CD200 receptors is provided in WO 00/70045 and WO 02/095030 and may also be obtained from GenBank (NM170780, NM138940, NM138939, NM138806, NT005612, XM293600, NM010818, AF497550, AF497549, AF497548, AF495380).

A CD200 or CD200R protein for use in modulating bone development may be prepared as a soluble fusion protein. The fusion protein may contain the extracellular domain of a CD200 or CD200R molecule linked to an immunoglobulin (Ig) Fc Region using techniques known in the art. Generally, a DNA sequence encoding the extracellular domain of a CD200 or CD200R molecule is linked to a DNA sequence encoding the Fc of the Ig and expressed in an appropriate expression system where the fusion protein is produced. The preparation of a CD200:Fc fusion protein is described in WO 99/24565 published May 20, 1999, which is incorporated herein by reference in its entirety.

The CD200 or CD200R peptide may also be modified to contain amino acid substitutions, insertions and/or deletions that do not alter the ability of the peptide to modulate bone development. Conserved amino acid substitutions involve replacing one or more amino acids of the amino acid sequence with amino acids of similar charge, size, and/or hydrophobicity characteristics. When only conserved substitutions are made the resulting analog should be functionally equivalent to the CD200 or CD200R peptide. Non-conserved substitutions involve replacing one or more amino acids of the amino acid sequence with one or more amino acids which possess dissimilar charge, size, and/or hydrophobicity characteristics.

The CD200 or CD200R protein may be modified to make it more therapeutically effective or suitable. For example, the protein or peptide thereof may be cyclized as cyclization allows a peptide to assume a more favourable conformation. Cyclization of peptides may be achieved using techniques known in the art. In particular, disulphide bonds may be formed between two appropriately spaced components having free sulfhydryl groups. The bonds may be formed between side chains of amino acids, non-amino acid components or a combination of the two. In addition, the CD200 or CD200R proteins or peptides of the present invention may be converted into pharmaceutical salts by reacting with inorganic acids including hydrochloric acid, sulphuric acid, hydrobromic acid, phosphoric acid, etc., or organic acids including formic acid, acetic acid, propionic acid, glycolic acid, lactic acid, pyruvic acid, oxalic acid, succinic acid, malic acid, tartaric acid, citric acid, benzoic acid, salicylic acid, benzenesulphonic acid, and toluenesulphonic acids.

### (b) Antibodies

Antibodies to CD200 or CD200 receptor proteins may be used as a CD200 receptor modulator. Antibodies to CD200 may act as a CD200 receptor antagonist by blocking the ability of CD200 to bind to the receptor. Whole antibodies to a CD200 receptor may act as a CD200R agonist by cross linking the receptor while antibody fragments to a CD200 receptor may act as a CD200R antagonist by blocking the ability of CD200 to bind to the receptor.

Antibodies that bind to a CD200 or CD200R protein or peptide can be prepared using techniques known in the art. Conventional methods can be used to prepare the antibodies. For example, by using a peptide of a CD200 or CD200 receptor polyclonal antisera or monoclonal antibodies can be made using standard methods. A mammal, (e.g., a mouse, hamster, or rabbit) can be immunized with an immunogenic form of the peptide which elicits an antibody response in the mammal. The amino acid sequence for CD200 and CD200R is known in the art and may be obtained from GenBank as described in part (a). Techniques for conferring immunogenicity on a peptide include conjugation to carriers or other techniques well known in the art. For example, the protein or peptide can be administered in the presence of adjuvant. The progress of immunization can be monitored by detection of antibody titers in plasma or serum. Standard ELISA or other immunoassay procedures can be used with the immunogen as antigen to assess the levels of antibodies. Following immunization, antisera can be obtained and, if desired, polyclonal antibodies isolated from the sera.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art, (e.g., the hybridoma technique originally developed by Kohler and Milstein (Nature 256, 495-497 (1975)) as well as other techniques such as the human B-cell hybridoma technique (Kozbor et al., Immunol. Today 4, 72 (1983)), the EBV-hybridoma technique to produce human monoclonal antibodies (Cole et al. Monoclonal Antibodies in Cancer Therapy (1985) Allen R. Bliss, Inc., pages 77-96), and screening of combinatorial antibody libraries (Huse et al., Science 246, 1275 (1989)). Hybridoma cells can be screened immunochemically for production of antibodies specifically reactive with the peptide and the monoclonal antibodies can be isolated. Therefore, the invention also contemplates hybridoma cells secreting monoclonal antibodies with specificity for CD200 or CD200R.

The term “antibody” as used herein is intended to include fragments thereof which also specifically bind with a CD200, CD200R or peptide thereof. Antibodies can be fragmented using conventional techniques and the fragments screened for utility in the same manner as described above. For example, F(ab′)₂ fragments can be generated by treating antibody with pepsin. The resulting F(ab′)₂ fragment can be treated to reduce disulfide bridges to produce Fab′ fragments.

Chimeric antibody derivatives, i.e., antibody molecules that combine a non-human animal variable region and a human constant region are also contemplated within the scope of the invention. Chimeric antibody molecules can include, for example, the antigen binding domain from an antibody of a mouse, rat, or other species, with human constant regions. Conventional methods may be used to make chimeric antibodies containing the immunoglobulin variable region which recognizes the gene product of a CD200R antigens of the invention (See, for example, Morrison et al., Proc. Natl. Acad. Sci. U.S.A. 81,6851 (1985); Takeda et al., Nature 314, 452 (1985), Cabilly et al., U.S. Pat. No. 4,816,567; Boss et al., U.S. Pat. No. 4,816,397; Tanaguchi et al., European Patent Publication EP171496; European Patent Publication 0173494, United Kingdom patent GB 2177096B). It is expected that chimeric antibodies would be less immunogenic in a human subject than the corresponding non-chimeric antibody.

Monoclonal or chimeric antibodies specifically reactive with a protein of the invention as described herein can be further humanized by producing human constant region chimeras, in which parts of the variable regions, particularly the conserved framework regions of the antigen-binding domain, are of human origin and only the hypervariable regions are of non-human origin. Such immunoglobulin molecules may be made by techniques known in the art, (e.g., Teng et al., Proc. Natl. Acad. Sci. U.S.A., 80, 7308-7312 (1983); Kozbor et al., Immunology Today, 4, 7279 (1983); Olsson et al., Meth. Enzymol., 92, 3-16 (1982)), and PCT Publication WO92/06193 or EP 0239400). Humanized antibodies can also be commercially produced (Scotgen Limited, 2 Holly Road, Twickenham, Middlesex, Great Britain.)

Specific antibodies, or antibody fragments, reactive against CD200 or CD200R proteins may also be generated by screening expression libraries encoding immunoglobulin genes, or portions thereof, expressed in bacteria with peptides produced from the nucleic acid molecules of CD200 or CD200R. For example, complete Fab fragments, VH regions and FV regions can be expressed in bacteria using phage expression libraries (See for example Ward et al., Nature 341, 544-546: (1989); Huse et al., Science 246, 1275-1281 (1989); and McCafferty et al. Nature 348, 552-554 (1990)). Alternatively, a SCID-hu mouse, for example the model developed by Genpharm, can be used to produce antibodies or fragments thereof.

### (c) Antisense Oligonucleotides

Antisense oligonucleotides that can modulate the expression and/or activity of a CD200 or CD200R may also be CD200 receptor modulators. Such antisense oligonucleotides may act as CD200 receptor antagonists.

The term “antisense oligonucleotide” as used herein means a nucleotide sequence that is complementary to its target.

The term “oligonucleotide” refers to an oligomer or polymer of nucleotide or nucleoside monomers consisting of naturally occurring bases, sugars, and intersugar (backbone) linkages. The term also includes modified or substituted oligomers comprising non-naturally occurring monomers or portions thereof, which function similarly. Such modified or substituted oligonucleotides may be preferred over naturally occurring forms because of properties such as enhanced cellular uptake, or increased stability in the presence of nucleases. The term also includes chimeric oligonucleotides which contain two or more chemically distinct regions. For example, chimeric oligonucleotides may contain at least one region of modified nucleotides that confer beneficial properties (e.g. increased nuclease resistance, increased uptake into cells), or two or more oligonucleotides of the invention may be joined to form a chimeric oligonucleotide.

The antisense oligonucleotides of the present invention may be ribonucleic or deoxyribonucleic acids and may contain naturally occurring bases including adenine, guanine, cytosine, thymidine and uracil. The oligonucleotides may also contain modified bases such as xanthine, hypoxanthine, 2-aminoadenine, 6-methyl, 2-propyl and other alkyl adenines, 5-halo uracil, 5-halo cytosine, 6-aza uracil, 6-aza cytosine and 6-aza thymine, pseudo uracil, 4-thiouracil, 8-halo adenine, 8-aminoadenine, 8-thiol adenine, 8-thioallyl adenines, 8-hydroxyl adenine and other 8-substituted adenines, 8-halo guanines, 8-amino guanine, 8-thiol guanine, 8-thioalkyl guanines, 8-hydroxyl guanine and other 8-substituted guanines, other aza and deaza uracils, thymidines, cytosines, adenines, or guanines, 5-trifluoromethyl uracil and 5-trifluoro cytosine.

Other antisense oligonucleotides of the invention may contain modified phosphorous, oxygen heteroatoms in the phosphate backbone, short chain alkyl or cycloalkyl intersugar linkages or short chain heteroatomic or heterocyclic intersugar linkages. For example, the antisense oligonucleotides may contain phosphorothioates, phosphotriesters, methyl phosphonates, and phosphorodithioates. In an embodiment of the invention there are phosphorothioate bonds links between the four to six 3′-terminus bases. In another embodiment phosphorothioate bonds link all the nucleotides.

The antisense oligonucleotides of the invention may also comprise nucleotide analogs that may be better suited as therapeutic or experimental reagents. An example of an oligonucleotide analogue is a peptide nucleic acid (PNA) wherein the deoxyribose (or ribose) phosphate backbone in the DNA (or RNA), is replaced with a polyamide backbone which is similar to that found in peptides (P. E. Nielsen, et al Science 1991, 254, 1497). PNA analogues have been shown to be resistant to degradation by enzymes and to have extended lives in vivo and in vitro. PNAs also bind stronger to a complementary DNA sequence due to the lack of charge repulsion between the PNA strand and the DNA strand. Other oligonucleotides may contain nucleotides containing polymer backbones, cyclic backbones, or acyclic backbones. For example, the nucleotides may have morpholino backbone structures (U.S. Pat. No. 5,034,506). Oligonucleotides may also contain groups such as reporter groups, a group for improving the pharmacokinetic properties of an oligonucleotide, or a group for improving the pharmacodynamic properties of an antisense oligonucleotide. Antisense oligonucleotides may also have sugar mimetics.

The antisense nucleic acid molecules may be constructed using chemical synthesis and enzymatic ligation reactions using procedures known in the art. The antisense nucleic acid molecules of the invention or a fragment thereof, may be chemically synthesized using naturally occurring nucleotides or variously modified nucleotides designed to increase the biological stability of the molecules or to increase the physical stability of the duplex formed with mRNA or the native gene e.g. phosphorothioate derivatives and acridine substituted nucleotides. The antisense sequences may be produced biologically using an expression vector introduced into cells in the form of a recombinant plasmid, phagemid or attenuated virus in which antisense sequences are produced under the control of a high efficiency regulatory region, the activity of which may be determined by the cell type into which the vector is introduced.

The antisense oligonucleotides may be introduced into tissues or cells using techniques in the art including vectors (retroviral vectors, adenoviral vectors and DNA virus vectors) or physical techniques such as microinjection. The antisense oligonucleotides may be directly administered in vivo or may be used to transfect cells in vitro which are then administered in vivo. In one embodiment, the antisense oligonucleotide may be delivered to macrophages and/or endothelial cells in a liposome formulation.

### (d) Peptide Mimetics

Peptide mimetics of a CD200 or CD200 receptor protein may also be prepared as CD200R modulators. Such peptides may include competitive inhibitors, enhancers, peptide mimetics, and the like. All of these peptides as well as molecules substantially homologous, complementary or otherwise functionally or structurally equivalent to these peptides may be used for purposes of the present invention.

“Peptide mimetics” are structures which serve as substitutes for peptides in interactions between molecules (See Morgan et al (1989), Ann. Reports Med. Chem. 24:243-252 for a review). Peptide mimetics include synthetic structures which may or may not contain amino acids and/or peptide bonds but retain the structural and functional features of a peptide, or enhancer or inhibitor of the invention. Peptide mimetics also include peptoids, oligopeptoids (Simon et al (1972) Proc. Natl. Acad, Sci USA 89:9367); and peptide libraries containing peptides of a designed length representing all possible sequences of amino acids corresponding to a peptide of the invention.

Peptide mimetics may be designed based on information obtained by systematic replacement of L-amino acids by D-amino acids, replacement of side chains with groups having different electronic properties, and by systematic replacement of peptide bonds with amide bond replacements. Local conformational constraints can also be introduced to determine conformational requirements for activity of a candidate peptide mimetic. The mimetics may include isosteric amide bonds, or D-amino acids to stabilize or promote reverse turn conformations and to help stabilize the molecule. Cyclic amino acid analogues may be used to constrain amino acid residues to particular conformational states. The mimetics can also include mimics of inhibitor peptide secondary structures. These structures can model the 3-dimensional orientation of amino acid residues into the known secondary conformations of proteins. Peptoids may also be used which are oligomers of N-substituted amino acids and can be used as motifs for the generation of chemically diverse libraries of novel molecules.

Peptides derived from the CD200 or CD200 receptor isoforms may also be used to identify lead compounds for drug development. The structure of the peptides described herein can be readily determined by a number of methods such as NMR and X-ray crystallography. A comparison of the structures of peptides similar in sequence, but differing in the biological activities they elicit in target molecules can provide information about the structure-activity relationship of the target. Information obtained from the examination of structure-activity relationships can be used to design either modified peptides, or other small molecules or lead compounds that can be tested for predicted properties as related to the target molecule. The activity of the lead compounds can be evaluated using assays similar to those described herein.

Information about structure-activity relationships may also be obtained from co-crystallization studies. In these studies, a peptide with a desired activity is crystallized in association with a target molecule, and the X-ray structure of the complex is determined. The structure can then be compared to the structure of the target molecule in its native state, and information from such a comparison may be used to design compounds expected to possess.

### (e) Screening Assays

The present invention also includes screening assays for identifying agents that modulate CD200 receptors and that are useful in modulating bone development. Agents that modulate include agents that stimulate a CD200 receptor (CD200R agonists) and agents that inhibit a CD200 receptor (CD200R antagonists).

In accordance with one embodiment, the invention enables a method for screening candidate compounds for their ability to modulate the activity of a CD200 receptor. The method comprises providing an assay system for assaying the outcome of CD200 receptor signalling, assaying the signalling activity in the presence or absence of the candidate or test compound and determining whether the compound has increased or decreased CD200 receptor signalling.

Accordingly, the present invention provides a method for identifying a compound that modulates bone development comprising:

(a) incubating a test compound with a bone cell expressing a CD200 receptor; and

(b) determining the effect of the compound on the CD200 receptor activity or expression and comparing with a control (i.e. in the absence of the test compound), wherein a change in the CD200 receptor activity or expression as compared to the control indicates that the test compound may modulate bone development.

A change in CD200 receptor activity includes a change in signalling through the receptor or a change in function that is associated with signalling through the receptor in a bone cell. For example, as described herein, stimulating a CD200 receptor can result in the increased expression of mRNA of molecules associated with bone metabolism as well as the release of cytokines that are associated with bone development. Therefore, one can measure cytokine levels and/or mRNA levels of certain molecules to determine whether or not CD200 receptor activity is modulated by a test compound. One can also use in vivo models. For example, the effects of CD200:CD200R modulators can be assessed in the ovariectomized rat model of osteoporosis. A full description of the protocol is given in Rixon et al, J. Bone & Mineral Research 9, 1179-1189, 1994 and Whitfield et al Calcif. Tissue hit. 58, 81-87, 1996 incorporated herein by reference. Briefly, normal, Sham-OVX (ovariectomized), and OVX Sprague-Dawley rats (3 months-old; 255-260 g) are randomized into groups. The animals receive 6, once-daily subcutaneous injections/week starting at the end of the second week after OVX, and ending at the end of the 8th week after OVX (i.e., 36 injections). Sham-OVX and OVX control rats receive 36 injections of vehicle (0.15 M NaCl containing 0.001N HCl) while OVX rats receive a selected dose of agent in vehicle. At the end of the 8th week after OVX, femurs are removed isolated, cleaned, and cut in half at mid-diaphysis and the proximal half is discarded. After removing the epiphysis, each half-femur is split lengthwise into two parts and the bone marrow is flushed out.

The bone-building potencies of the test compounds are assessed from the changes in the mean thicknesses (area/perimeter) of the trabecular in the distal half-femurs from the variously treated animals. To measure mean trabecular thickness, the two demineralized half-femurs from each rat are dehydrated and embedded in paraffin. Longitudinal, 10-μm sections from the middle plane of each bone are cut and then strained with Sanderson's rapid bone stain. The mean trabecular thickness is measured using an imaging system. An increase in the values for trabecular thickness for the drug-treated group.

One can also use an in vitro assays for testing the effect of a test compound on bone development. For example, the assay that is sold commercially by Millenium Biologix as “Osteologic Disks” can be used. This assay provides a thin film of calcium phosphate (hydroxyapatite) material that, when used to plate osteoclasts, reveals resorption by those osteoclasts. The slides with plated osteoclasts therefore can be used to identify agents that inhibit osteoclast-mediated resorption, revealed as a reduction in the number or area that the osteoclasts create in the film, relative to a control in which no agent is present.

In one embodiment, the screening assays of the invention can be used to identify CD200 receptor agonists. The term “CD200 receptor agonist” as used herein means any agent that can bind, crosslink or ligate a CD200 receptor and result in the stimulation of the receptor. Stimulating a CD200 receptor includes stimulating the signalling through the receptor or stimulating a function of a bone cell that is associated with signalling through the receptor. For example, the stimulation of the receptor by the agonist will result in a net increase in bone development, by way of a reduction in osteoclastogenesis and a reduction in the consequent bone resorption mediated by the osteoclasts.

Accordingly, the present invention provides a method of identifying a CD200R agonist useful in stimulating bone development comprising the steps of:

(a) incubating a test compound with a bone cell expressing a CD200R; and

(b) determining whether or not the test compound stimulates a CD200R, wherein stimulation of the CD200R indicates that the test compound is a CD200R agonist that may be useful in stimulating bone development.

In a specific assay, one can test the ability of the test compound to bind to a CD200R on a bone cell. Accordingly, the present invention provides a method of identifying compounds which bind with a CD200 receptor, comprising the steps of:

(a) incubating a bone cell expressing a CD200 receptor and a test compound, under conditions which allow for formation of a complex, and

(b) assaying for complexes of the CD200 receptor and the test compound, for free compound, and for non-complexed CD200 receptor, wherein the presence of complexes indicates that the test compound is capable of binding the CD200 receptor.

In another embodiment, the screening assay can be used to identify CD200 receptor antagonists. The term “CD200 receptor antagonist” as used herein means any agent that can inhibit the activation or stimulation of a CD200 receptor. Inhibiting a CD200 receptor includes inhibiting signalling through the receptor and inhibiting a function of a cell that is associated with the receptor.

Accordingly, the present invention provides a screening assay for identifying an antagonist of a CD200 receptor useful in inhibiting bone development comprising the steps of:

(a) incubating a test compound with a CD200 receptor; and

(b) determining whether or not the test compound inhibits the CD200 receptor, wherein inhibition of the CD200R indicates that the compound is a CD200R antagonist and may be useful in inhibiting bone development.

One skilled in the art will appreciate that many methods can be used in order to determine whether or not a test compound can inhibit a CD200R and inhibit bone development.

In another embodiment, an assay can be developed to identify modulators of bone development by testing for the effect of the test compound on the interaction of a CD200R agonist and CD200R on a bone cell. Accordingly, the present invention provides a screening assay for identifying a modulator of bone development comprising the steps of:

(a) incubating a test compound with a bone cell having a CD200 receptor;

(b) adding a CD200 receptor agonist; and

(c) determining whether or not the test compound modulates bone development.

One of skill in the art will appreciate that many methods can be used to test the effect of the test compound on bone development. In one embodiment, one can measure the mRNA levels of one or more molecules associated with bone development such as OPG:OPGL, TRAP, RANK, BSP, OC and Cbfa-1 in the treated bone cells. One can also measure the levels of one or more cytokines in the culture medium such as IL-1, IL-6, TFGβ and TNFα. Methods for measuring the mRNA and cytokine levels are described in greater detail in Example 1.

In all of the above screening assays, the test compound can be any compound which one wishes to test including, but not limited to, proteins, peptides, nucleic acids (including RNA, DNA, antisense oligonucleotide, peptide nucleic acids), carbohydrates, organic compounds, small molecules, natural products, library extracts, bodily fluids and other samples that one wishes to test for modulators of a CD200 receptor.

The CD200 receptor is expressed on the surface of a bone cell in the above assays. The bone cell used in the screening assay can be any bone cell that expresses a CD200R or a bone cell that has been transfected with a CD200R. Types of bone cells that may be used include, but are not limited to, fresh cultured cells or cell lines including osteoblast progenitor cells, osteoblasts, osteoclasts, osteocytes, chondroclasts, chondrocytes, chondroclasts or bone marrow cells, including mesenchymal stem and progenitor cells. In addition, a number of bone cell lines are available commercially including, but not limited to, MC3T3-E1 cells, MG-63 cells, U2OS cells, UMR-106 cells, ROS 17/2.8 cells and SaOS-2 cells (see the American Type Culture Collection (ATCC) catalog).

The screening methods of the invention include high-throughput screening applications. For example, a high-throughput screening assay may be used which comprises any of the methods according to the invention wherein aliquots of bone cells transfected with a CD200 receptor are exposed to a plurality of test compounds within different wells of a multi-well plate. Further, a high-throughput screening assay according to the invention involves aliquots of transfected cells which are exposed to a plurality of candidate factors in a miniaturized assay system of any kind. Another embodiment of a high-throughput screening assay could involve exposing a transfected cell population simultaneously to a plurality of test compounds.

The method of the invention may be “miniaturized” in an assay system through any acceptable method of miniaturization, including but not limited to multi-well plates, such as 24, 48, 96 or 384-wells per plate, micro-chips or slides. The assay may be reduced in size to be conducted on a micro-chip support, advantageously involving smaller amounts of reagent and other materials. Any miniaturization of the process which is conducive to high-throughput screening is within the scope of the invention.

The invention extends to any compounds or modulators of a CD200 receptor identified using the screening method of the invention that are useful in modulating bone development.

The invention also includes a pharmaceutical composition comprising a modulator of a CD200 receptor identified using the screening method of the invention in admixture with a suitable diluent or carrier. The invention further includes a method of preparing a pharmaceutical composition for use in modulating bone development comprising mixing a modulator of a CD200 receptor identified according to the screening assay of the invention with a suitable diluent or carrier.

The present invention also includes all business applications of the screening assay of the invention including conducting a drug discovery business. Accordingly, the present invention also provides a method of conducting a drug discovery business comprising:

(a) providing one or more assay systems for identifying a modulator of a CD200 receptor;

(b) conducting therapeutic profiling of modulators identified in step (a), or further analogs thereof, for efficacy and toxicity in animals; and

(c) formulating a pharmaceutical preparation including one or more modulators identified in step (b) as having an acceptable therapeutic profile.

In certain embodiments, the subject method can also include a step of establishing a distribution system for distributing the pharmaceutical preparation for sale, and may optionally include establishing a sales group for marketing the pharmaceutical preparation.

The present invention also provides a method of conducting a target discovery business comprising:

(a) providing one or more assay systems for identifying modulators of a CD200 receptor;

(b) (optionally) conducting therapeutic profiling of modulators identified in step (a) for efficacy and toxicity in animals; and

(c) licensing, to a third party, the rights for further drug development and/or sales for modulators identified in step (a), or analogs thereof.

### III. Pharmaceutical Compositions

The present invention includes pharmaceutical compositions containing one or more modulators of a CD200R. Accordingly, the present invention provides a pharmaceutical composition for use in modulating bone development comprising an effective amount of a CD200R modulator in admixture with a suitable diluent or carrier.

In one embodiment, the present invention provides a pharmaceutical composition for use in stimulating bone development comprising an effective amount of a CD200R agonist in admixture with a suitable diluent or carrier.

In another embodiment, the present invention provides a pharmaceutical composition for use in inhibiting bone development comprising an effective amount of a CD200R antagonist in admixture with a suitable diluent or carrier.

Such pharmaceutical compositions can be for intralesional, intravenous, topical, rectal, parenteral, local, inhalant or subcutaneous, intradermal, intramuscular, intrathecal, transperitoneal, oral, and intracerebral use. The composition can be in liquid, solid or semisolid form, for example pills, tablets, creams, gelatin capsules, capsules, suppositories, soft gelatin capsules, gels, membranes, tubelets, solutions or suspensions. The CD200 receptor or ligand is preferably injected in a saline solution either intravenously, intraperitoneally or subcutaneously. In the alternative, and for localized delivery rather than systemic delivery, for instance for the treatment of bone fracture and for dental applications, the selected therapeutic agent can be formulated as a paste or as a hardened cement using such bone compatible matrix materials as hydroxyapatite, and generally any calcium phosphate material that will set either before or after its application to a bone site such as a fracture or void. The ambient-setting cements are preferred, so that conditions likely to denature the selected therapeutic are avoided.

The pharmaceutical compositions of the invention can be intended for administration to humans or animals. Dosages to be administered depend on individual needs, on the desired effect and on the chosen route of administration.

The pharmaceutical compositions can be prepared by per se known methods for the preparation of pharmaceutically acceptable compositions which can be administered to patients, and such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985).

On this basis, the pharmaceutical compositions include, albeit not exclusively, the active compound or substance in association with one or more pharmaceutically acceptable vehicles or diluents, and contained in buffered solutions with a suitable pH and iso-osmotic with the physiological fluids. The pharmaceutical compositions may additionally contain other immune modulatory agents.

A pharmaceutical composition comprising the nucleic acid molecules of the invention may be used in gene therapy to modulate bone development. Recombinant molecules comprising a nucleic acid sequence encoding a CD200 or CD200R molecule of the invention, or fragment thereof, may be directly introduced into cells or tissues in vivo using delivery vehicles such as retroviral vectors, adenoviral vectors and DNA virus vectors. They may also be introduced into cells in vivo using physical techniques such as microinjection and electroporation or chemical methods such as coprecipitation and incorporation of DNA into liposomes. Recombinant molecules may also be delivered in the form of an aerosol or by lavage. The nucleic acid molecules of the invention may also be applied extracellularly such as by direct injection into cells. The nucleic acid molecules encoding a CD200 or CD200R molecule are preferably prepared as a fusion with a nucleic acid molecule encoding an immunoglobulin (Ig) Fc region. As such, the CD200 or CD200R molecule will be expressed in vivo as a soluble fusion protein.

The above pharmaceutical compositions may include other immune modulatory molecules such as cytokines, MD-1 or fgl2.

The following non-limiting examples are illustrative of the present invention:

### EXAMPLE

### Example 1

### Materials and Methods

Mice: C57BL/6 mice, along with breeder pairs of mice constructed with homologous deletion (KO) of IL-1^{r} or TNFα^{r} (p55,75), were purchased from the Jackson Laboratories, Bar Harbour, Me. Male mice (6-8 weeks of age) were used as cell donors throughout.
Cell cultures: Stromal cells were obtained from 5-day cultures of bone marrow from control or KO mice, and cultured (1×10⁴ cells/well) in osteologic slides (Millenium Biologix, Kingston, Canada), with/without deliberate addition of 1×10² MC3T3 osteoblastic cells/well. Note that gene expression in cultured osteoblast lines alters over time in culture (21) as determined by cDNA microarray analysis, potentially reflecting changes responsible for a reduction in bone regeneration in older osteoblasts. Accordingly all studies reported herein were performed using MC3T3 cells frozen at P24, and used within 7 days of thawing and transfer to fresh medium.

In some experiments cells were incubated in the presence of exogenous CD200Fc (22), or anti-CD200R (23) as a means of modifying bone cell development. Medium was changed at 2-day intervals, and included M-CSF, dexamethasone (10⁻⁸M), ascorbate (75 μg/ml), -glycerol phosphate (10 mM) and 0.5% normal mouse serum. In most studies, 2 days prior to harvest, cells were pulsed with 0.5% serum from LPS injected mice (10 mg/mouse ip 24 hrs earlier). In studies noted in the text cells on osteologic slides were incubated in a humidified CO₂ incubator in order to allow access to culture supernatants (for cytokine protein analysis by ELISA-see below). All slides were fixed at day 10 in RNA-later (Ambion), mRNA harvested in TRizol solution, and a comparison made between ground and simulated microgravity cultures using real-time PCR.

Real-time PCR: Primer pairs were designed in all cases to detect ˜100 bp amplicons for the genes of interest. Gene expression in real-time PCR was normalized to a composite of the geometric mean expression of 3 housekeeping genes (GAPDH, Transferrin^{r} and β-actin), to account for the >100-fold variability in expression even in housekeeping genes (see Figures).
Cytokine analysis: Culture supernatants (50 μl) were harvested from individual wells of osteologic slides at completion of culture and assessed for IL-1, IL-6, TGFβ, TNFα and IL-1ra levels using ELISA assays and commercial cytokine-specific mAbs obtained from Pharmingen (San Diego, Calif.). Plates were precoated with capture antibody (100 ng/ml) and developed with 50 ng/ml of biotinylated developing antibody. Streptavidin-coupled alkaline phosphatase with appropriate substrate was used to develop the assay, and recombinant mouse cytokines (Endogen, San Diego, Calif.) were used to quantitate the assay. The following antibodies were used: PM425B1 and MM425BB, for IL-1β; goat polyclonal Ig, Q19, and biotinylated M20 (Santa Cruz Biotechnology Inc., Santa Cruz, Calif.), for IL-1ra; MP5-20F3 and MP5-32C11, for IL-6; R4-6A2 and XMG1.2 for IFNγ; G281-2626 and MP6-XT3 for TNFα.

### Results

mRNA expression in bone cultures following osteoclast activation by serum cytokines:

In preliminary studies, the inventor examined both steady-state mRNA levels of molecules associated with the regulation of bone metabolism in bone marrow stromal cells grown in the presence/absence of the osteoblast progenitor cells MC3 T3, following treatment (in the last 48 hrs of culture) with a cytokine mixture present in the serum of LPS-treated mice. Cytokine production in the supernatant of these cultures was also assessed.

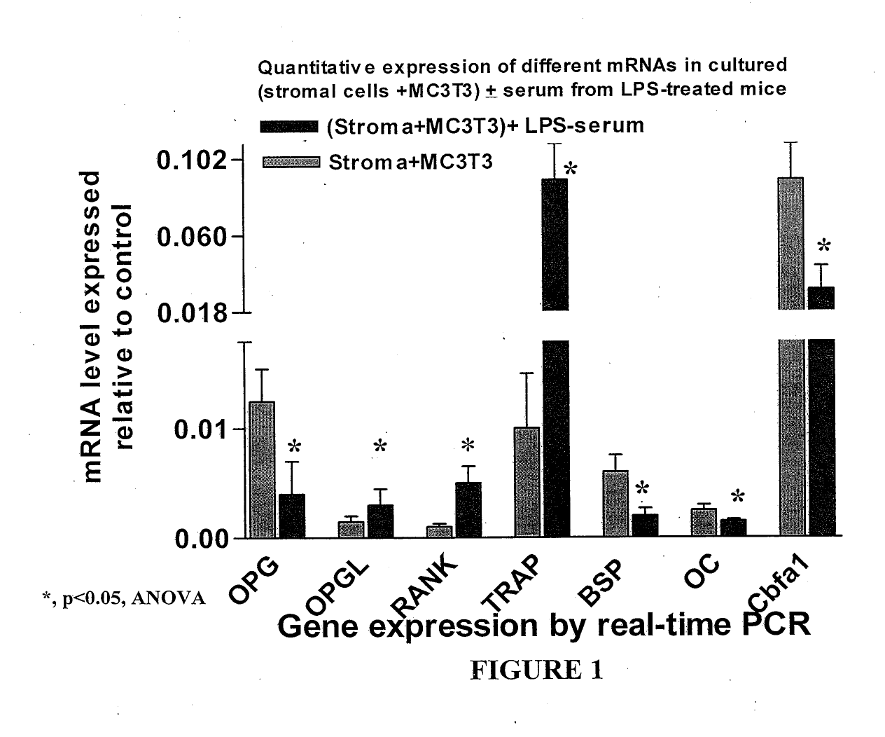

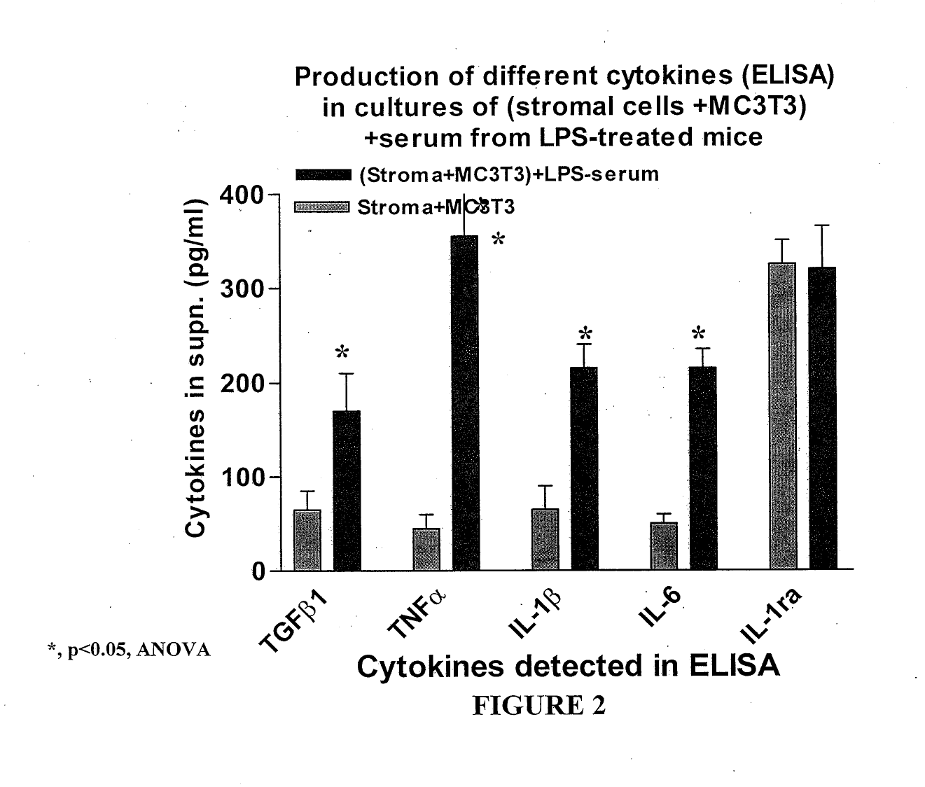

As indicated in FIGS. 1 and 2, LPS-treatment led to a marked increased expression of mRNAs associated with osteoclastogenesis in such cultures (increased TRAP, RANK, decreased OPG:OPGL-see Table 1), and a corresponding fall in markers of osteoclastogenesis (BSP, OC and Cbfa1)-see FIG. 1. When cytokines were measured, increased expression of IL-1, IL-6 and TNFα were most marked relative to control cultures, with decreased IL-1ra:IL-1 ratios (Table 1).

Comparison of mRNA expression in bone cultures in the presence/absence of anti-CD200R:

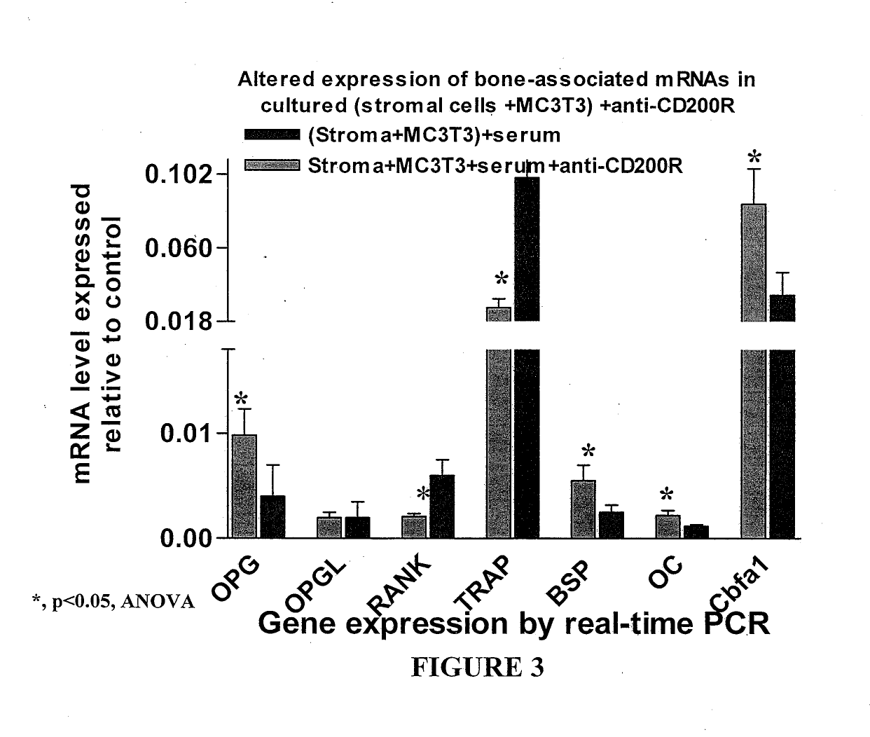

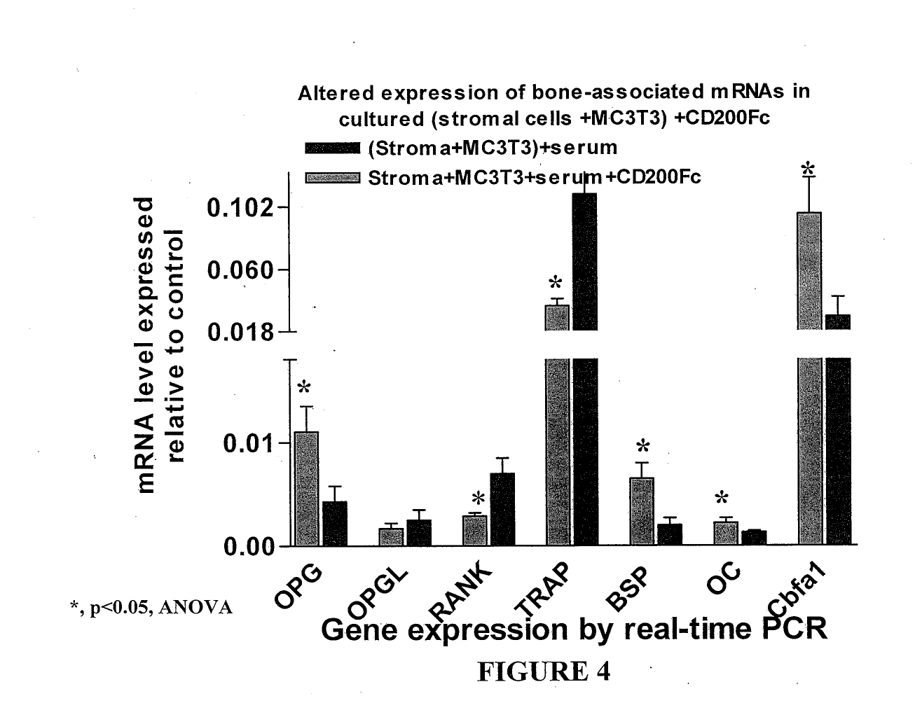

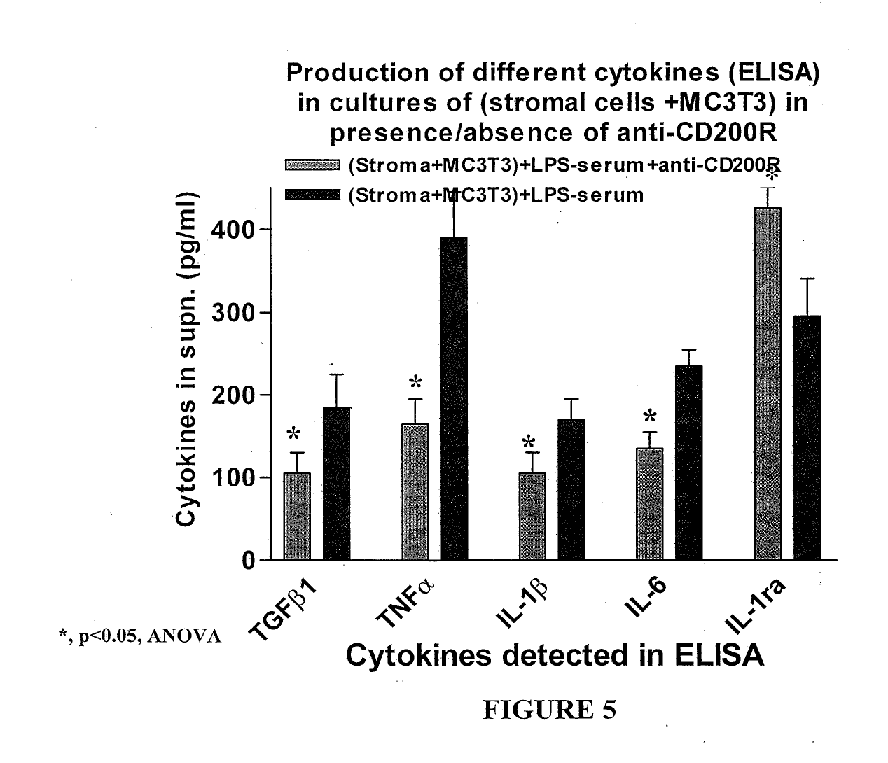

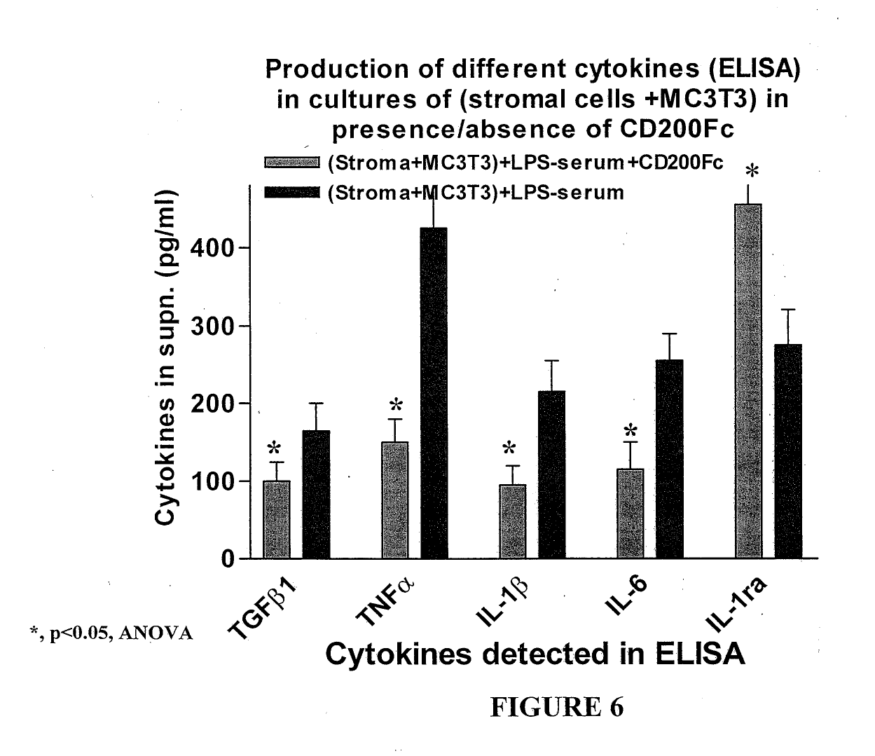

The inventor next assessed steady-state mRNA levels of various molecules and cytokines implicated in the regulation of bone metabolism in bone marrow stromal cells grown in the presence/absence of CD200Fc or anti-CD200R mAbs, again with cells stimulated during the last 48 hrs of culture with serum from LPS-treated mice (a source of cytokines known to induce increased osteoclast activity-see FIG. 1). Cytokines, and the variety of more bone-specific mRNAs (e.g. BSP, OC, OPG etc.) were quantitated as described in the Materials and Methods by ELISA or real-time PCR, using a composite of various mRNAs as internal standard. Comparative data for mRNA expression in one of three such studies are shown in FIGS. 3 and 4 (anti-CD200R, CD200Fc respectively). In addition, supernatants were harvested from these same cultures and IL-1, IL-6, TGFβ1, TNFα and MCP-1 assayed by ELISA (FIGS. 5 and 6).

It is evident from these studies that incubation in the presence of either anti-CD200R or CD200Fc halted the increased expression of mRNAs for markers of osteoclastogenesis in these cultures (relative to controls-see FIG. 1), with now evidence for increased bone sialoprotein and osteocalcin, as well as the (osteoblastic) transcription factor Cbaf1. Consistent with these findings the ratio of OPG:OPGL was markedly elevated in the presence of both anti-CD200R and CD200Fc (minimal change in OPG with decreased OPGL), as indicated in Table 1. With the concomitant decrease in RANK mRNA expression, these changes would be expected to result in decreased osteoclastogenesis. The decreased TRAP mRNA expression is similarly consistent with this hypothesis.

Comparison of Cytokine Protein Production from Anti-CD200R Stimulated Bone Marrow Stromal Cells:

Data analyzing cytokine/chemokine protein production in the supernatants of cultures described for FIGS. 3 and 4 are shown in FIGS. 5 and 6 (again one of 3 studies).

### Discussion

There are a number of recent reviews on the regulation of bone metabolism/differentiation by OPG:OPGL (6, 24, 25). These confirm that inhibition of OPGL in vivo via OPG decreases bone destruction and local bone resorption. Little TRAP mRNA is detected under conditions of increased OPG:OPGL, presumably reflecting the fact that bone destruction is generally due at least in part to the action of OCs activated by OPGL. These data in turn are consistent with evidence from OPG (OPGL) knockout mice respectively, showing increased susceptibility to osteopetrosis/osteoporosis (respectively), and from studies in collagen-induced arthritis models in rodents. Thus, in the latter regard, an Fc-osteoprotegerin fusion protein (Fc-OPG) was infused into rats following induction of CIA, and paraffin-embedded joints were analyzed histologically with the adjacent bone assessed by histomorphometry. OCs were identified using TRAP staining and expression of the mRNA for OPG and RANKL was identified by in situ hybridization. Short-term Fc-OPG effectively prevented joint destruction, despite having no impact on the inflammatory aspects of CIA, and in arthritic joints, OC numbers were decreased >75%, and bone erosion scores by >60%, by Fc-OPG(26).

Reciprocal regulation of the differentiation of myeloid precursors in bone marrow into OCs or dendritic cells (DCs) is mediated by M-CSF or GM-CSF respectively, in association with a number of other cytokines, neurohormones, and endocrinological factors (27). DC maturation is also inhibited when c-Fos is expressed at an early stage of differentiation (28), suggesting that c-Fos is a key mediator of the lineage commitment between OCs and DCs. Amongst the other transcriptionally activated genes following programming of OC differentiation are those encoding for a number of chemokine receptors, presumably involved in regulating chemotaxis of OCs to sites of bone turnover. The dominant chemokine receptor expressed by OCs is CCR1, followed by CCR3 and CX3CR1 (29). In contrast, a number of receptors expressed on macrophages and associated with inflammatory responses, including CCR2 and CCR5, were down-regulated by RANKL. CCL9, which acts through CCR1, was observed in these studies to stimulate cytoplasmic motility and polarization in OCs, in a fashion akin to that seen in response to CCL3/MIP-1α, which also acts through CCR1 and is chemotactic for OCs.

When myeloid cells develop into DCs there is evidence for a functionally important role for OPG and its ligands in regulating the interactions between T cells and DCs. DCs express RANK while T cells express RANKL, and the ligation of RANK by RANKL can activate both T cells and DCs. There is evidence that both B cells and DCs secrete OPG, and this secretion is regulated by the CD40 receptor (30). OPG (−/−) mice have B-cell developmental defects, and in addition, DCs isolated from these mice present antigen more efficiently in vitro and secrete elevated amounts of inflammatory cytokines when stimulated with LPS or soluble RANKL in vitro.

As noted above, a number of other factors are known to modulate bone differentiation, including parathyroid hormone (PTH), PGE2, inflammatory (and other) cytokines, and 1.25(OH)(2)-vitamin D-3. However, examination of OC/OB differentiation in cells derived from PGE-receptor knockout mice showed that in fact PTH increased RANKL and IL-6 and decreased OPG mRNA levels similarly in both wild-type and EP2−/− or EP4−/− cells, with the major defect in the response to PGE (2) in animals lacking either EP2 or EP4 receptors being a reduction in basal and stimulated RANKL levels (31). IL-4 abrogates osteoclastogenesis through STAT6-dependent inhibition of NF-kappa B (32), while IL-11 stimulates osteoclastic resorption in mouse Calvary by mechanisms that are partially dependent on PGE2, are sensitive to inhibition by IL-4, IL-13, and OPG, and are associated with enhanced expression of RANKL and OPG (33). Independently Brandstrom (34) has shown that OPG secretion from human marrow stromal cells is decreased by PGE2 and DEX, but increased by IL-1 and TNFα, with subsequent modulation of OC differentiation.

The stimulation of gene expression of OPGL in mouse OBs is activated by LPS acting on Toll-like receptors (35). It is of interest in this regard that CpG ODN (a ligand for TLR-9) inhibited RANKL-induced osteoclastogenesis when present from the initiation of bone marrow cultures, but increased RANKL-induced osteoclastogenesis in RANKL-pretreated cells. CpG ODN enhanced the expression of IL-1β and TNFα, and antibodies to TNFα or the TNF^{r}-type 1, (though not IL-1ra), blocked CpG ODN-induced osteoclastogenesis in RANKL-pretreated cultures (36). Interestingly, CpG ODN reduced expression of the M-CSF receptor, which is known to be important during the initiation of OC differentiation. These data suggest that CpG ODN, via down-modulation of M-CSF receptor, can inhibit early steps of OC differentiation, though through the induction of TNFα, it can simultaneously support osteoclastogenesis in cells that are committed to the OC differentiation pathway.

Cell culture studies with OB cell lines (e.g. MC3T3-E1), and/or co-culture studies of these cells with hematopoietic progenitor cells, have been a popular means to explore the complexity of OC/OB differentiation. Thus osteoblastic cell differentiation has been shown to be positively regulated by Notch (37), implying a potential unexpected novel role for this molecule in regulating osteoporosis. MC3T3-E1 cells are known constitutively to express BMP-2, BMP-4, and BMP-7, while Noggin, a specific BMP inhibitor, reversibly blocks ascorbate-induced gene expression associated with OB differentiation, indicating that BMP production by MC3T3-E1 cells was necessary for differentiation (20). Indeed, the ability of exogenously added BMP-2, BMP-4, or BMP-7 to stimulate osteocalcin (OCN) and bone sialoprotein (BSP) mRNAs or OCN promoter activity was synergistically increased in cells that were actively synthesizing an extracellular matrix (i.e., were grown in the presence of ascorbate-see also (38)). In other studies BMP-2 induced or enhanced the expression of the OB differentiation markers alkaline phosphatase (ALP) and osteocalcin (OC). In contrast, TGFβ1 was not only unable to induce these markers, but inhibited BMP-2-mediated OC gene expression, ALP activity and the ability of BMP-2 to induce MC3T3-E1 mineralization, all of which inhibitor functions were independent of Osf2/Cbfa1 gene expression (11).

Both OPG and TGFβ inhibited OC formation in hemopoietic cell/OB cocultures, but the kinetics of their action differed. TGFβ also inhibited osteoclastogenesis in cocultures of cells derived from OPG knockout mice (opg(−/−)). TGFβ decreased RANKL messenger RNA (mRNA) expression in cultured OBs, and addition of exogenous RANKL to TGFβ-inhibited cocultures of opg(−/−) cells partially restored osteoclastogenesis. These data suggest that the inhibitory actions of TGFβ were mediated mainly by decreased OB production of RANKL. In contrast, in the absence of OBs, TGFβ increased OC formation in recombinant RANKL- or TNFα-stimulated cultures of hemopoietic cells or even of RAW 264.7 macrophage-like cells many time beyond levels attainable by maximal stimulation by RANKL or TNFα alone, implying that TGFβ increases OC formation through an action on OC precursors (39).

Similarly, 1.25 (OH) (2) vitamin D-3-stimulated OC formation in spleen-OB cocultures is mediated in part at least indirectly by enhanced IL-1α and RANKL production in Obs (40). These effects of 1.25 (OH) (2)-vitamin D-3 on OB differentiation are further modulated in opposing manners by BMP7 (suppressive) and TGFβ1 (enhancing) (41).

A recent study by Kim and co-workers (42) is reminiscent of the work reported above documenting a role for CD200:CD200R interactions in further modulating bone development in vitro. This group characterized an OC-associated receptor (OSCAR) as a novel member of the leukocyte receptor complex (LRC)-encoded family which was expressed specifically in OCs. Genes in the LRC are known to produce immunoglobulin (Ig)-like surface receptors which have been reported to play important roles in the regulation of both innate and adaptive immune responses. Unlike other members of the LRC complex, however, OSCAR expression is restricted to preosteoclasts or mature OCs. Its putative-ligand (OSCAR-L) is expressed primarily in OBs and/or stromal cells. In a study paralleling that shown in FIG. 2, addition of a soluble form of OSCAR in coculture with osteoblasts inhibited the formation of OCs from bone marrow precursor cells in the presence of bone-resorbing factors, suggesting that OSCAR may be a bone-specific regulator of OC differentiation.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
OPG:OPGL (RT-PCR), and IL-1ra:IL-1 (ELISA) ratios in stromal cell cultures
(+MC3T3) incubated under various conditions, with/without serum from
LPS-treated mice, and anti-CD200R/CD200Fc

| | OPG:OPGL ratio | | | IL-1ra:IL-1 ratio | | |
|---|---|---|---|---|---|---|
| Cell culture under test^{a} | FIG. 1 | FIG. 3 | FIG. 4 | FIG. 2 | FIG. 5 | FIG. 6 |
| Control stroma + MC3T3 | 8.0 | | | 5.0 | | |
| Control stroma + MC3T3 + serum | 1.3* | 2.0* | 1.7* | 1.5* | 1.7* | 1.3* |
| Control stroma + MC3T3 + | | 4.9 | | | 4.0 | |
| serum + anti-CD200R | | | | | | |
| Control stroma + MC3T3 + | | 6.5 | | | 4.8 | |
| serum + CD200Fc | | | | | | |

Footnotes:
^{a}Data refer to cells cultured (i.e. stroma with MC3T3 cells), and conditions of culturing (simulated with/without serum from LPS-treated mice, and + anti-CD200R or CD200Fc).
b. Refers to Figures where detailed description of study/data can be found. SD < 25% in all cases.
*p < 0.05 compared with control cultures (no serum from LPS-treated mice)

1. Wooden, S., Bennett, L. and Boone, T., Cell 89, 309-319, 1997.
2. Lacey, D. L., Timms, E., Tan, H. L., Kelly, M. J., Dunstan, C. R., Burgress, T., Elliott, R., Colombero, A., Elliott, G. and Scully, S., Cell 93, 165-176, 1998.
3. Kong, Y. Y., Yoshida, H., Sarosi, I., Tan, H. L., Timms, E., Capparelli, C., Morony, S., Oliveira-dos-Santos, A. J., Van, G. and Itie, A., Nature 397, 315-323, 1999.
4. Takahashi, N., Udagawa, N. and Suda, T., Biochem Biophys Res Commun 256, 449-455, 1999.
5. Mori, K., Miyamoto, N., Higuchi, Y., Nanba, K., Ito, M., Tsurudome, M., Nishio, M., Kawano, M., Uchida, A. and Ito, Y., Cell Immunol 207, 118-126, 2001.
6. Hofbauer, L. C. and Heufelder, A. E., J Molecular Med Jmm 79, 243-253, 2001.
7. Khosla, S., Endocrinology 142, 5050-5055, 2001.
8. Kostenuik, P. J. and Shalhoub, V., Curr Pharm Design 7, 613-635, 2001.
9. Komine, M., Kukita, A., Kukita, T., Ogata, Y., Hotokebuchi, T. and Kohashi, O., Bone 28, 474-483, 2001.
10. Gilbert, L., He, X. F., Farmer, P., Boden, S., Kozlowski, M., Rubin, J. and Nanes, M. S., Endocrinology 141, 3956-3964, 2000.
11. SpinellaJaegle, S., RomanRoman, S., Faucheu, C., Dunn, F. W., Kawai, S., Gallea, S., Stiot, V., Blanchet, A. M., Courtois, B., Baron, R. and Rawadi, G., Bone 29, 323-330, 2001.
12. Thirunavukkarasu, K., Miles, R. R., Halladay, D. L., Yang, X. H., Galvin, R. J. S., Chandrasekhar, S., Martin, T. J. and Onyia, J. E., J Biol Chem 276, 36241-36250, 2001.
13. Dai, J. L., Lin, D. L., Zhang, J., Habib, P., Smith, P., Murtha, J., Fu, Z., Yao, Z., Qi, Y. H. and Keller, E. T., J Clin Invest 106, 887-895, 2000.
14. Frost, A., Jonsson, K. B., Brandstrom, H., Ljunghall, S., Nilsson, O. and Ljunggren, O., Bone 28, 268-274, 2001.
15. Kon, T., Cho, T. J., Aizawa, T., Yamazaki, M., Nooh, N., Graves, D., Gerstenfeld, L. C. and Einhom, T. A., J Bone Miner Res 16, 1004-1014, 2001.
16. Takayanagi, H., Ogasawara, K., Hida, S., Chiba, T., Murata, S., Sato, K., Takaoka, A., Yokochi, T., Oda, H. and Tanaka, K., Nature 408, 600-605, 2000.
17. Srivastava, S., Toraldo, G., Weitzmann, M. N., Cenci, S., Ross, F. P. and Pacifici, R., J Biol Chem 276, 8836-8840, 2001.
18. Cenci, S., Weitzmann, M. N., Roggia, C., Namba, N., Novack, D., Woodring, J. and Pacifici, R., J Clin Invest 106, 1229-1237, 2000.
19. Halladay, D. L., Miles, R. R., Thirunavukkarasu, K., Chandrasekhar, S., Martin, T. J. and Onyia, J. E., J Cell Biochem 84, 1-11, 2002.
20. Xiao, G. Z., Gopalakrishnan, R., Jiang, D., Reith, E., Benson, M. D. and Franceschi, R. T., J Bone Miner Res 17, 101-110, 2002.
21. Huang, W. B., Carlsen, B., Rudkin, G. H., Shah, N., Chung, C., Ishida, K., Yamaguchi, D. T. and Miller, T. A., in “Biochem Biophys Res Commun,”, 2001.
22. Gorczynski, R. M., Cattral, M. S., Chen, Z. G., Hu, J. A., Lei, J., Min, W. P., Yu, G. and Ni, J., J Immunol 163, 1654-1660, 1999.
23. Gorczynski, R. M., Eur J Immunol 31, 2331-2337, 2001.
24. Kermanach, N. S., Bessis, N., CohenSolal, M., DeVernejoul, M. C. and Boissier, M. C., Eur Cytokine Netw 13, 144-153, 2002.
25. Jones, D. H., Kong, Y. Y. and Penninger, J. M., Ann Rheum Dis 61, 32-39, 2002.
26. Romas, E., Sims, N. A., Hards, D. K., Lindsay, M., Quinn, J. W. M., Ryan, P. F. J., Dunstan, C. R., Martin, T. J. and Gillespie, M. T., Amer J Pathol 161, 1419-1427, 2002.
27. Cappellen, D., LuongNguyen, N. H., Bongiovanni, S., Grenet, O., Wanke, C. and Susa, M., J Biol Chem 277, 21971-21982, 2002.
28. Miyamoto, T., Ohneda, 0., Arai, F., Iwamoto, K., Okada, S., Takagi, K., Anderson, D. M. and Suda, T., Blood 98, 2544-2554, 2001.
29. Lean, J. M., Murphy, C., Fuller, K. and Chambers, T. J., J Cell Biochem 87, 386-393, 2002.
30. Bengtsson, A. K. and Ryan, E. J., Crit. Rev Immun 22, 201-215, 2002.
31. Li, X., Pilbeam, C. C., Pan, L., Breyer, R. M. and Raisz, L. G., Bone 30, 567-573, 2002.
32. AbuAmer, Y., J Clin Invest 107, 1375-1385, 2001.
33. Ahlen, J., Anderson, S., Mukohyama, H., Roth, C., Backman, A., Conaway, H. H. and Lerner, U. H., Bone 31, 242-251, 2002.
34. Li, L., Khansari, A., Shapira, L., Graves, D. T. and Amar, S., Infec Immunity 70, 3915-3922, 2002.
35. Kikuchi, T., Matsuguchi, T., Tsuboi, N., Mitani, A., Tanaka, S., Matsuoka, M., Yamamoto, G., Hishikawa, T., Noguchi, T. and Yoshikai, Y., J Immunol 166, 3574-3579, 2001.
36. Zou, W., Schwartz, H., Endres, S., Hartmann, G. and BarShavit, Z., Faseb J 16, 274-282, 2002.
37. Tezuka, K., Yasuda, M., Watanabe, N., Morimura, N., Kuroda, K., Miyatani, S, and Hozumi, N., JBone Miner Res 17, 231-239, 2002.
38. DAlonzo, R. C., Kowalski, A. J., Denhardt, D. T., Nickols, G. A. and Partridge, N. C., J Biol Chem 277, 24788-24798, 2002.
39. Quinn, J. M. W., Itoh, K., Udagawa, N., Hausler, K., Yasuda, H, Shima, N., Mizuno, A., Higashio, K., Takahashi, N., Suda, T., Martin, T. J. and Gillespie, M. T., J Bone Miner Res 16, 1787-1794, 2001.
40. Lee, S. K., Kalinowski, J., Jastrzebski, S, and Lorenzo, J. A., J Immunol 169, 2374-2380, 2002.
41. Eichner, A., Brock, J., Heldin, C. H. and Souchelnytskyi, S., J Exp Cell Res 275, 132-142, 2002.
42. Kim, N., Takami, M., Rho, J., Josien, R. and Choi, Y., J Exp Med 195, 201-209, 2002.
43. Langdahl, et al 2002, J. Bone Min. Res. 17: 1245-1255.

1-56. (canceled)

57: A method of modulating bone development comprising administering an effective amount of an agent that modulates a CD200 receptor to a cell or animal in need thereof.

58: A method according to claim 57 comprising administering an effective amount of a CD200 receptor agonist to stimulate bone development.

59: A method according to claim 58 wherein the CD200 receptor agonist is selected from the group consisting of antibodies, peptide mimetics, small molecules, CD200 proteins and fragments thereof, soluble CD200, CD200 receptor proteins and fragments thereof, and soluble CD200 receptors.

60: A method according to claim 58 wherein the CD200 receptor agonist is a CD200 protein or fragment thereof.

61: A method according to claim 58 wherein the CD200 receptor agonist is an antibody that cross-links a CD200 receptor.

62: A method according to claim 58 for the treatment or prevention of a condition or disease associated with increased osteoclastogenesis or bone loss.

63: A method according to claim 62 wherein the bone loss is associated with inflammatory conditions, infection, injury, genetic disorders and aging.

64: A method according to claim 62 wherein the disease or condition is osteoporosis, osteogenesis imperfecta, Paget's disease, metastatic bone cancer, myeloma bone disease, or bone fracture healing.

65: A method according to claim 57 comprising administering an effective amount of CD200 receptor antagonist to inhibit bone development.

66: A method according to claim 65 wherein the antagonist is selected from the group consisting of an antibody fragment, small molecule, peptide mimetic, peptide or an antisense oligonucleotide to a CD200 receptor.

67: A method according to claim 65 for the treatment or prevention of a disease or condition associated with decreased osteoclastogenesis or increased bone mass.

68: A method according to claim 67 wherein the disease or condition is associated with osteopetrosis or fibrous dysplasia.

69: A method for identifying a compound that modulates bone development comprising:
(a) incubating a test compound with a bone cell expressing a CD200 receptor; and
(b) determining the effect of the compound on the CD200 receptor activity or expression and comparing with a control, wherein a change in the CD200 receptor activity or expression as compared to the control indicates that the test compound may modulate bone development.

70: A method of identifying a CD200R agonist useful in stimulating bone development comprising the steps of:
(a) incubating a test compound with a bone cell expressing a CD200R; and
(b) determining whether or not the test compound stimulates a CD200R, wherein stimulation of the CD200R indicates that the test compound is a CD200R agonist that may be useful in stimulating bone development.

71: A screening assay for identifying an antagonist of a CD200 receptor useful in inhibiting bone development comprising the steps of:
(a) incubating a test compound with a CD200 receptor; and
(b) determining whether or not the test compound inhibits the CD200 receptor, wherein inhibition of the CD200R indicates that the compound is a CD200R antagonist and may be useful in inhibiting bone development.

72: A modulator of bone development comprising the steps of:
(a) incubating a test compound with a bone cell having a CD200 receptor;
(b) adding a CD200 receptor agonist; and
(c) determining whether or not the test compound modulates bone development.

73: A method of identifying substances which bind with a CD200 receptor, comprising the steps of:
(a) incubating a bone cell expressing a CD200 receptor and a test substance, under conditions which allow for formation of a complex, and
(b) assaying for complexes of the CD200 receptor and the test substance, for free substance, and for non-complexed CD200 receptor, wherein the presence of complexes indicates that the test substance is capable of binding the CD200 receptor.
